(19) United States
(12) Patent Application Publication
(10) Pub. No.: US 2019/0117202 A1
(43) Pub. Date: Apr. 25, 2019

(54) DEVICES AND METHODS FOR AUTOMATED FILLING AND DISPENSING OF ADIPOSE TISSUE WITH CONTROL OF SHEAR

(71) Applicant: LifeCell Corporation, Branchburg, NJ (US)

(72) Inventors: Kai-Roy Wang, Jersey City, NJ (US); Christopher Locke, Bournemouth (GB); Dennis Y. Lee, Scotch Plains, NJ (US); Evan J. Friedman, Montvale, NJ (US); Israel Jessop, Annandale, NJ (US); Sangwook Park, Dunellen, NJ (US)

(73) Assignee: LifeCell Corporation, Branchburg, NJ (US)

(21) Appl. No.: 15/772,601

(22) PCT Filed: Nov. 1, 2016
(86) PCT No.: PCT/US2016/059870

§ 371 Date: May 1, 2018

Related U.S. Application Data
(60) Provisional application No. 62/249,536, filed on Nov. 2, 2015.

(51) Int. Cl. A61B 10/02; A61M 5/142

(52) U.S. Cl. CPC A61B 10/0283; A61M 1/0009; A61M 2202/08; A61M 5/14212

(57) ABSTRACT

Devices, systems, and methods for tissue transfer are disclosed that can allow control of transfer speed. The devices use positive and negative pressure to advance a plunger (322) into or out of a body (311) of the device. A pressure regulator may be used to control the applied pressure. Maintaining transfer speed within acceptable ranges and/or controlling pressure or shear forces on tissues can improve the viability of certain types of tissue.

This application claims priority to U.S. Provisional Patent Application No. 62/249,536, filed Nov. 2, 2015, the entire contents of which is incorporated herein by reference.

The present disclosure relates to surgical instruments and methods including instruments and methods for transfer of tissue such as adipose tissue.

Autologous fat grafting has become increasingly common and has numerous clinical applications such as facial contouring, breast reconstruction and/or augmentation, and other aesthetic or reconstructive procedures. In addition, autologous fat grafting has been found to have relatively low donor-site morbidity compared with other surgical options.

In some cases, however, autologous fat grafting provides somewhat unpredictable outcomes. For example, the amount of adipose cell viability after implantation is variable, which can result in less than optimal outcomes and/or require multiple or revision procedures.

The reasons for the unpredictability in fat-graft outcomes are not completely understood. Some clinicians, however, have found a correlation between aspects of the surgical procedures used and ultimate graft viability. For example, J. H. Lee et al. have studied the correlations between aspiration pressure during graft collection, injection pressure, and sheer stress on graft viability. J. H. Lee et al., “The Effect of Pressure and Shear on Autologous Fat Grafting,” Plastic and Reconstructive Surgery, May 2003: 1125-1136. Lee concluded that higher aspiration and injection pressures, up to a point, did not affect fat graft viability in vivo, but the degree of shear stress, which is a function of flow rate, did significantly affect fat graft viability. In addition, fat grafts injected slowly with low shear stress outperformed grafts injected with high shear stress. Id.

Adipocyte viability can be affected by a number of factors including aspiration pressure, injection pressure, and sheer stress. If done improperly, the loading and unloading of cells from syringes and other vessels can result in damage to the cells and reduce overall cell viability after implantation. To mitigate these effects, the user must carefully control pressures and sheer stresses when loading and unloading tissues. This control can be achieved by introducing a level of automation and repeatability in cell transfer.

Various instruments have been described to assist surgeons in controlling the amount of pressure or shear applied to fat grafts during collection and reinjection. For example, US Patent Publication Number 2013/0158515 A1 by Austen describes systems with sensors to measure and/or control pressure, shear, and injection velocity. Similarly, US Patent Publication Number 2012/0209248 describes systems for collection and injection of adipose tissue, which allow control of injection pressure below certain limits. These systems, however, have some limitations.

The present disclosure provides devices and methods for improved tissue transfer, including devices and methods for transferring adipose tissue. The devices and methods allow controlled loading and unloading of adipose delivery devices and can reduce operative times while controlling tissue transfer processes to increase or control the consistency of cell viability during tissue transfer.

In certain embodiments, a tissue transfer device is provided. The device includes a body including a chamber. An interior portion of the body is adapted to accept at least a portion of a plunger of a syringe. The device also includes an inlet in fluid communication with an interior portion of the chamber. A positive or negative pressure applied at the inlet causes the plunger of the syringe to advance into or out of the interior portion of the body.

In some embodiments, a tissue transfer device is provided. The device comprises a body including a chamber including an outer wall and an interior portion contained within the outer wall. The device also includes a plunger contained at least partially within the interior portion. The device also includes an inlet in fluid communication with an interior portion of the chamber. The device is configured such that a positive or negative pressure applied at the inlet causes the plunger to move within the interior portion of the body.

In some embodiments, a tissue handling system is provided. The system includes a syringe and a tissue transfer device. The syringe includes a syringe body having an interior volume and including a peripheral wall. The syringe also includes a syringe plunger disposed within the syringe body. The syringe also includes a syringe flange surrounding at least a portion of the peripheral wall. The tissue transfer device includes a body including a chamber. An interior portion of the body is adapted to accept at least a portion of the plunger of the syringe. The tissue transfer device also includes an inlet in fluid communication with an interior portion of the chamber. A positive or negative pressure applied at the inlet causes the plunger of the syringe to advance into or out of the interior portion of the body of the device.

In certain embodiments, a method of transferring tissue is provided. The method includes selecting a tissue transfer device having a body including a chamber and an inlet. An interior portion of the body is adapted to accept at least a portion of a plunger of a syringe. The inlet is in fluid communication with an interior portion of the chamber. The method also includes coupling the plunger of the syringe to the tissue transfer device. The method also includes applying a negative pressure at the inlet to cause the plunger of the syringe to advance into the interior portion of the body. The method also includes an optional step of applying a positive pressure at the inlet to cause the plunger of the syringe to advance out of the interior portion of the body.

### DESCRIPTION OF THE DRAWINGS

FIG. 1 depicts a tissue transfer device according to various embodiments.

FIGS. 2A and 2B depicts a tissue transfer device and a tissue transfer device coupled to a syringe, respectively, according to various embodiments.

FIG. 3 depicts a tissue handling system including a tissue transfer device and a syringe according to various embodiments.

FIG. 4 depicts the system of FIG. 3 in a different state of tissue loading and unloading.

FIG. 5 depicts a tissue transfer device that can accommodate multiple tissue receptacles according to various embodiments.

FIG. 6 depicts a tissue transfer device according to various embodiments.

FIG. 7 depicts a tissue transfer device according to various embodiments.

FIG. 8 depicts a method of transferring tissue according to various embodiments.

### DESCRIPTION OF CERTAIN EXEMPLARY EMBODIMENTS

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The use of the word “syringe” is not limited to any industry standard and includes any of a variety of receptacles in different shapes and sizes. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Although the present instruments and methods are described specifically for transfer or injection of adipose tissues, it will be appreciated that the devices and methods may be used with other suitable materials including other tissue types or products that may be subject to damage by excess pressure and/or shear or would benefit from the automated transfer processes described herein. Further, the present device may be used to facilitate transfer or injection of other substances (e.g., medications, tissue fillers, dyes, contrast agents, or fluids), when control of the pressure or maintenance of transfer speed may be important for appropriate delivery and/or to prevent damage to an implantation site. Systems for control of shear forces on adipose tissue are described, for example, in U.S. patent application Ser. No. 14/682,342 filed on Apr. 9, 2015, the entire contents of which is incorporated herein by reference.

A tissue transfer device is presented that facilitates loading and unloading of tissues or fluids. The tissue transfer device can employ air pressure or vacuum to adjust the position of a plunger or piston in a tissue receptacle. The use of regulated air pressure can allow automated loading and unloading and can improve predictability, repeatability, and graft success in adipose (or other tissue) transfer.

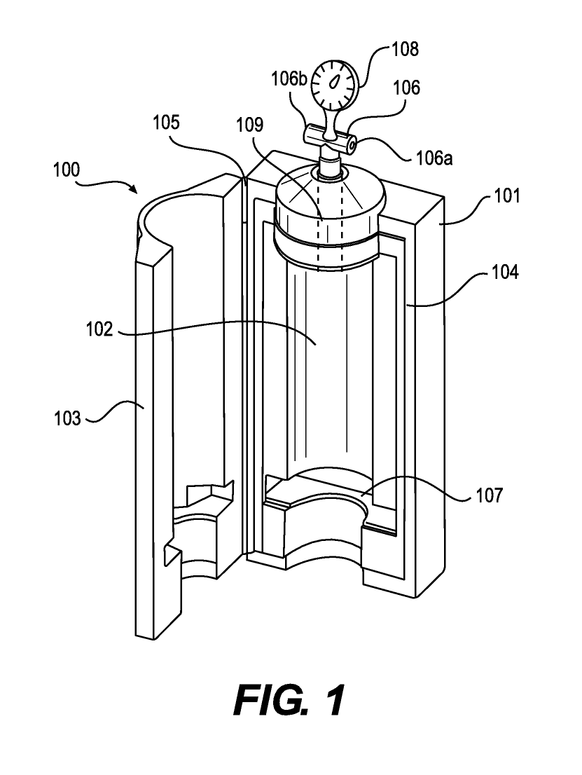

FIG. 1 depicts a tissue transfer device 100 according to various embodiments. The tissue transfer device 100 can include a body 101 that includes a chamber 102. An inlet 106 is in fluid communication with the interior volume of the chamber 102. When the tissue transfer device 100 is coupled to a tissue receptacle such as a syringe, a change in the air pressure inside the chamber 102 can move an element of the tissue receptacle such as a plunger or piston.

The body 101 of the tissue transfer device 100 can be made of any suitable material that meets application-specific requirements. Such materials can include, but are not limited to, plastics, metals, and ceramics. In some embodiments, the body can include separate pieces, and one of the pieces may be a lid 103 or closure. The attachment mechanism between the lid 103 and other pieces of the body 101 can include hinges 105 of various designs. Alternatively, the lid 103 can attach to other pieces of the body 101 using a sliding friction fit or other suitable attachment methods.

In accordance with various embodiments, the body 101 can include a sealing gasket 104 that creates a seal around at least a portion of the chamber 102. In some embodiments, the sealing gasket 104 can be an o-ring or other structure made of various non-porous materials or one or more non-porous surfaces that press together to form a seal. To facilitate the seal, the body 101 may include a clasp or locking mechanism to hold the lid 103 tight to other pieces of the body 101. In some embodiments, reduced air pressure inside the chamber 102 can facilitate initial sealing at the sealing gasket 104 by holding fast the pieces of the body 101 including the lid 103. The sealing gasket 104 can form a seal among the pieces of the body 101 or between and among the body 101, lid 103, and elements of a tissue receptacle such as a syringe including a syringe body. The interior of the body 101 can include a recess 107 that is shaped to accept at least a portion of a tissue receptacle. In some embodiments, the recess 107 is shaped to accommodate a flange of a syringe. The recess 107 can stabilize the main portion of a tissue receptacle (such as a body) and hold it motionless as the piston or plunger moves due to changes in air pressure inside the chamber 102.

The inlet 106 can be disposed at any location on the body 101 that does not interfere with passage of a piston or plunger through the body 101. In one embodiment, the inlet 106 is located on the body 101 opposite to the entry point of a plunger or piston in a similar arrangement to intake and exhaust valves in a standard engine cylinder. The inlet 106 can be connected directly to the chamber 102 or can be in fluid communication with the chamber via a lumen 109.

The inlet 106 can include one or more ports 106a, 106b. In accordance with various embodiments, the port(s) 106a, 106b may be shaped or terminated to facilitate connection of pressure or vacuum sources. For example, the port(s) 106a, 106b could be a plastic through-port, a luer-type connector, a threaded connector, a swage fitting, or a pressure-fit connector. The pressure source attached to a port 106a, 106b can include a pressurized gas canister, a house source of medical compressed gas provided by a facility, or a mechanical pump. The vacuum source attached to a port 106a, 106b can include a mechanical pump or house vacuum provided by a facility.

In one embodiment, a pressure regulator 108 is placed in the line between or otherwise connected to the pressure or vacuum source and the inlet 106. The pressure regulator 108 can control the pressure to allow smooth motion of the piston or plunger within the body 101—preventing excessive shear forces on the tissue, which is known to reduce cell viability. In some embodiments, the pressure regulator 108 can be designed to include preset pressures for different sized tissue receptacles or cannulas or for different procedures. For example, the pressure regulator 108 may be set to 31 psi or less when the tissue receptacle is a 60 cubic centimeter syringe. The use of an air pressure/vacuum source and pressure regulator 108 can create a constant level of pressure/vacuum in the chamber 102 that, in turn, provides continuous motion of the piston or plunger throughout a transfer operation. In other words, avoiding fluctuations in pressure in the chamber 102 can prevent unwanted changes in velocity of the piston or plunger particularly at the beginning or end of a tissue transfer operation.

In one embodiment, the pressure regulator 108 of the device 100 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied within the chamber 102 of the device 100. The memory of the computing device may include lookup tables or processor-executable instructions to ascertain a safe operating pressure range based on the user input(s). In some embodiments, the computing device may prevent a user from exceeding a preset maximum flow velocity and/or shear rate.

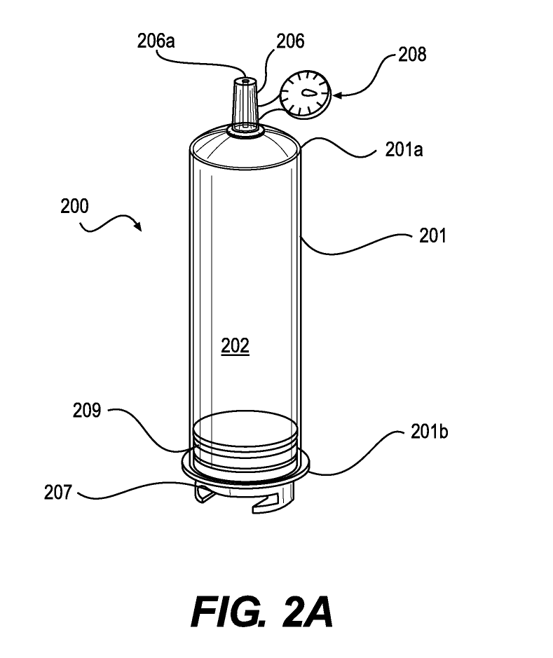

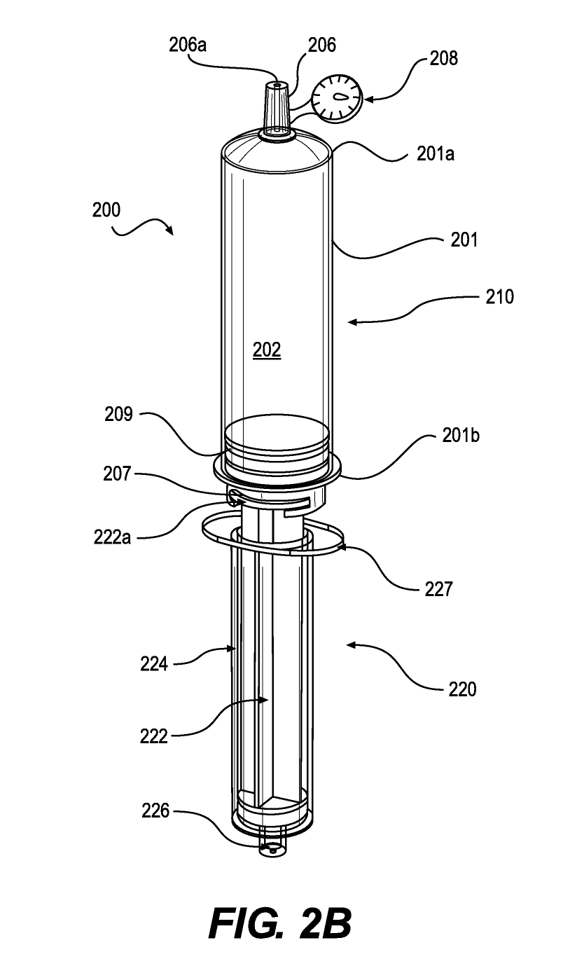

An alternative embodiment of a tissue transfer device 200 is depicted in FIGS. 2A and 2B. The device 200 can include a body 201 having a proximal end 201a, a distal end 201b, and a chamber 202. The device 200 can also include an inlet 206 and a stopper 209 that may be coupled to a mechanical adaptor 207. Changing the air pressure within the chamber 202 using either a high or low pressure source can cause the stopper 209 to move within the body 201. When the stopper 209 is attached or otherwise couple (e.g., by suction) to a tissue receptacle such as a syringe using the mechanical adaptor 207, the motion of the stopper 209 can cause a piston or plunger of the tissue receptacle to advance into or out from the interior of the body 201 thereby drawing tissue into or expelling tissue from the tissue receptacle. In the embodiment shown in FIG. 2B, a syringe 220 can be coupled to a tissue transfer device 200. The syringe may include a body 224, a flange 227, an outlet 226, and a syringe plunger 222 having a head 222a.

The body 201 of the tissue transfer device 200 can be made of any suitable material that meets application-specific requirements. Such materials can include, but are not limited to, plastics, metals, and ceramics. In some embodiments, the body 201 of the tissue transfer device 200 may be transparent. In accordance with various embodiments, the diameter of the body 201 of the tissue transfer device 200 is approximately equal to a maximum diameter of a head of the piston or plunger. In some embodiments, the diameter of the body 201 of the device 200 is larger than a maximum diameter of the head of the piston or plunger or a body of the tissue receptacle to thereby increase the filling or injection force. Increased filling or injection forces can improve efficiency of operation with respect to extremely viscous fluids.

In accordance with various embodiments, the body 201 can include a plunger 209 that creates a seal at an end of the chamber 202. In some embodiments, the plunger 209 can include an o-ring made of various non-porous materials or may include a non-porous surface that presses against an inner wall of the body 201 to form a seal. In accordance with various embodiments, the plunger 209 can be attached to a mechanical adaptor 207. The mechanical adaptor 207 can attach to an element of the tissue receptacle to cause the element to move in concert with motion of the plunger 209. In an exemplary embodiment, the mechanical adaptor 207 can engage with a head of a piston or plunger 222 for a syringe as depicted in FIG. 2B. Engagement of the mechanical adaptor 207 with an element of the tissue receptacle can utilize a shape fit, friction fit, adhesives, interlocking elements, fasteners, or any other suitable engagement system as dictated by application-specific requirements.

A proximal end 201b of the device 200 can engage with a portion of the tissue receptacle to stabilize the receptacle during a tissue loading operation. In some embodiments, a flange of a syringe can abut the proximal end 201b of the device 200 to prevent movement of the syringe body. For example, the flange 227 of a syringe 220 can abut the proximal end 201b of the device 200 as shown in FIG. 2B. In some embodiments, the device 200 includes additional mounting elements such as straps, adhesives, or complementary threading that can engage the tissue receptacle and prevent movement or separation of the receptacle from the device 200.

The inlet 206 can be disposed at any location on the body 201 that does not interfere with passage of the plunger 209 through the body 201. In one embodiment, the inlet 206 is located on a proximal end 201b of the body 201 opposite the distal end 201a. The inlet 206 can be connected directly to the chamber 202 or can be in fluid communication with the chamber via a lumen.

The inlet 206 can include one or more ports 206a. In accordance with various embodiments, the port(s) 206a may be shaped or terminated to facilitate connection of pressure or vacuum sources. For example, the port(s) 206a could be a plastic through-port, a luer-type connector, a threaded connector, a swage fitting, or a pressure-fit connector. As described above with reference to the embodiment of FIG. 1, the pressure source attached to a port 206a can include a pressurized gas canister, a house source of medical compressed gas provided by a facility, or a mechanical pump. The vacuum source attached to a port 206a can include a mechanical pump or house vacuum provided by a facility.

In some embodiments, a pressure regulator 208 is placed in the line between or otherwise couple with the pressure or vacuum source and the inlet 206. The pressure regulator 208 can provide control of pressure to allow smooth motion of the piston or plunger within the body 201. Control of pressure or vacuum level prevents excessive shear forces on the tissue, which is known to reduce cell viability. In some embodiments, the pressure regulator 208 can be designed to include preset pressures for different sized tissue receptacles or cannulas or for different procedures. For example, the pressure regulator may be set to 31 psi or less when the tissue receptacle is a 60 cc syringe. The use of an air pressure/vacuum source and pressure regulator 208 can create a constant level of pressure/vacuum in the chamber 202 that, in turn, provides continuous motion of the piston or plunger throughout a transfer operation. In other words, avoiding fluctuations in pressure in the chamber 202 can prevent unwanted changes in velocity of the piston or plunger particularly at the beginning or end of a tissue transfer operation.

In an embodiment, the pressure regulator 208 of the device 200 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied within the chamber 202 of the device 200. The memory of the computing device may include lookup tables or processor-executable instructions to ascertain a safe operating pressure range based on the user input(s). In some embodiments, the computing device may prevent a user from exceeding a preset maximum flow velocity and/or shear rate.

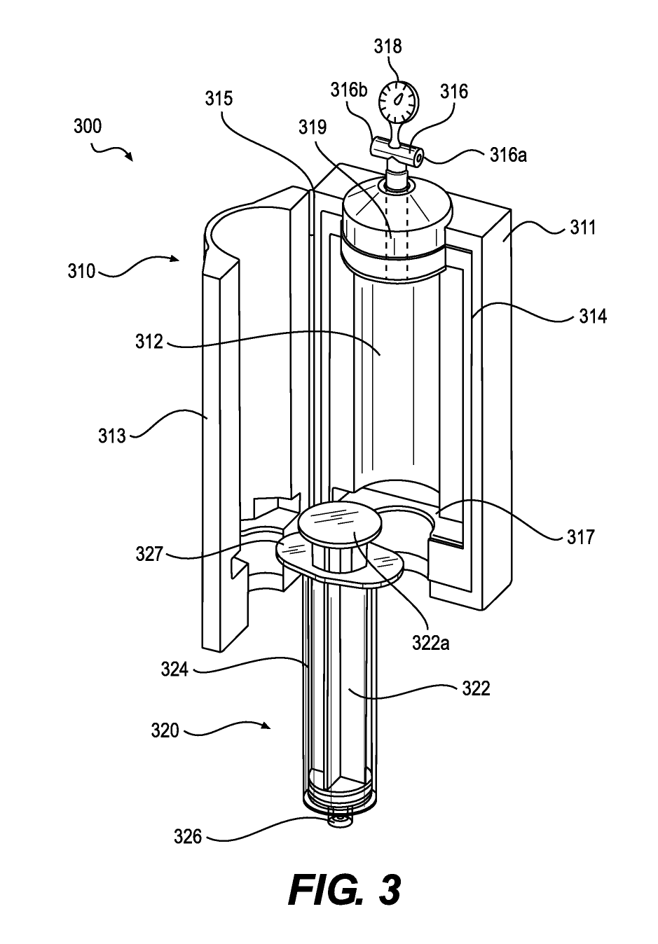

A tissue handling system according to various embodiments is depicted in FIG. 3. The tissue handling system 300 can include a tissue transfer device 310 and a syringe 320. The tissue transfer device 310 can include a body 311 that includes a chamber 312. An inlet 316 can be in fluid communication with the interior volume of the chamber 312. The syringe 320 can include a syringe body 324, a syringe plunger 322, and an inlet 326 to receive tissue. When the tissue transfer device 310 is coupled to the syringe 320, a change in air pressure inside the chamber 312 can move syringe plunger 322 thereby drawing a tissue into or expelling a tissue from the syringe body 324.

The body 311 of the tissue transfer device 310 can be made of any suitable material that meets application-specific requirements. Such materials can include, but are not limited to, plastics, metals, and ceramics. In some embodiments, the body can include separate pieces, and one of the pieces may be a lid 313. The attachment mechanism between the lid 313 and other pieces of the body 311 can include hinges 315 of various designs. Alternatively, the lid 313 can attach to other pieces of the body 311 using a sliding friction fit or other suitable attachment methods.

In accordance with various embodiments, the body 311 can include a sealing gasket 314 that creates a seal around at least a portion of the chamber 312. In some embodiments, the sealing gasket 314 can be an o-ring made of various non-porous materials or one or more non-porous surfaces that press together to form a seal. To facilitate the seal, the body 311 may include a clasp or locking mechanism to hold the lid 313 tight to other pieces of the body 311. In some embodiments, reduced air pressure inside the chamber 312 can facilitate initial sealing at the sealing gasket 314 by holding fast the pieces of the body 311 including the lid 313. The sealing gasket 314 can form a seal among the pieces of the body 311 or between and among the body 311, lid 313, and elements of a tissue receptacle such as a syringe including a syringe body. The interior of the body 311 can include a recess 317 that is shaped to accept at least a portion of a tissue receptacle. In some embodiments, the recess 317 is shaped to accommodate a syringe flange 327. In various embodiments, the recess 317 can stabilize the syringe flange 327 or syringe body 324 and hold it motionless as syringe plunger 322 moves due to changes in air pressure inside the chamber 312.

The inlet 316 can be disposed at any location on the body 311 that does not interfere with passage of a piston or plunger through the body 311. In a preferred embodiment, the inlet 316 is located on the body 311 opposite to the entry point of a plunger or piston in a similar arrangement to intake and exhaust valves in a standard engine cylinder. The inlet 316 can be connected directly to the chamber 312 or can be in fluid communication with the chamber via a lumen 319.

The inlet 316 can include one or more ports 316a, 316b. In accordance with various embodiments, the port(s) 316a, 316b may be shaped or terminated to facilitate connection of pressure or vacuum sources. For example, the port(s) 316a, 316b could be a plastic through-port, a luer-type connector, a threaded connector, a swage fitting, or a pressure-fit connector. The pressure source attached to a port 316a, 316b can include a pressurized gas canister, a house source of medical compressed gas provided by a facility, or a mechanical pump. The vacuum source attached to a port 316a, 316b can include a mechanical pump or house vacuum provided by a facility. In preferred embodiments, a pressure regulator 318 is placed in the line between the pressure or vacuum source and the inlet 316. The pressure regulator 318 can provide a steady and reliable level of high or low pressure to allow smooth motion of the piston or plunger within the body 311. The use of a set pressure or vacuum level prevents excessive shear forces on the tissue, which is known to reduce cell viability. In some embodiments, the pressure regulator 318 can be designed to include preset pressures for different sized syringes 320 or cannulas or for different procedures. For example, the pressure regulator 318 may be set to 31 psi or less when syringe 320 is a 60 cc syringe. The use of an air pressure/vacuum source and pressure regulator 318 can create a constant level of pressure/vacuum in the chamber 312 that, in turn, provides continuous motion of the piston or plunger throughout a transfer operation. In other words, avoiding fluctuations in pressure in the chamber 312 can prevent unwanted changes in velocity of the syringe piston 322 particularly at the beginning or end of a tissue transfer operation.

In an embodiment, the pressure regulator 318 of the device 310 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied within the chamber 312 of the device 310. The memory of the computing device may include lookup tables or processor-executable instructions to ascertain a safe operating pressure range based on the user input(s). In some embodiments, the computing device may prevent a user from exceeding a preset maximum flow velocity and/or shear rate.

The syringe body 324 can have a variety of sizes and a range of inner volumes. A syringe flange 327 can be attached to the syringe body 324. The syringe flange 327 may surround the entire syringe body 324 or may only project from the body 324 at a few locations. The syringe inlet 326 can be coupled to a needle or cannula to allow injection of material collected in the syringe body 324.

The syringe plunger 322 can include a head 322a. In accordance with various embodiments, a diameter of the head 322a can be approximately equal to an inner diameter of the body 311 of the tissue transfer device 310. When the plunger 322 advances into the interior of the body 311, a vacuum is created in the interior 325 of the syringe body 324 that pulls tissue or fluid into the interior 325 through the inlet 326.

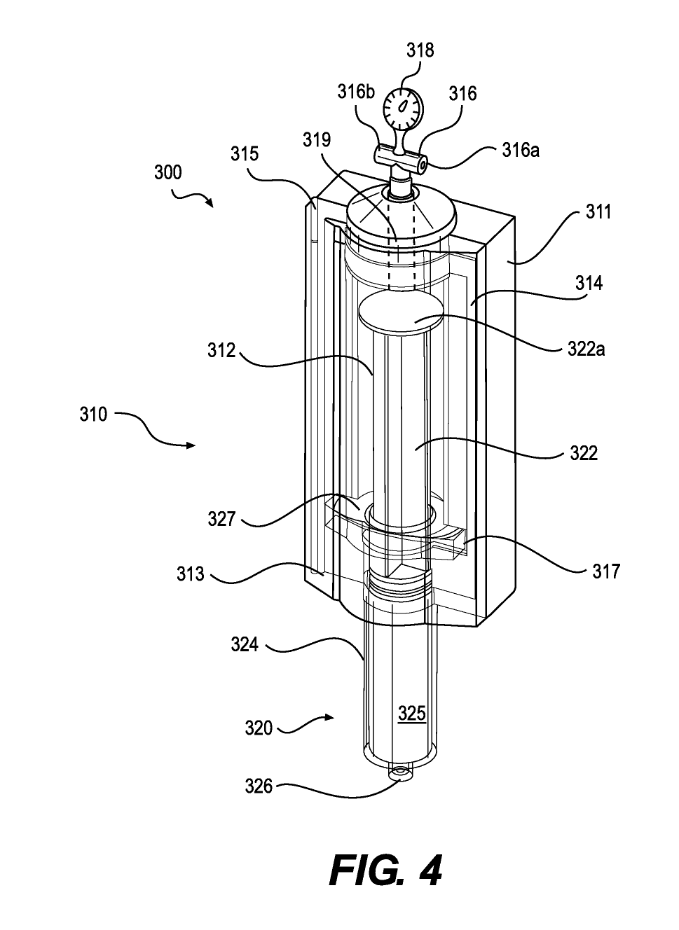

The system 300 is depicted in FIG. 4 after a filling operation has completed. In this figure, the syringe 320 has been coupled to the tissue transfer device 310. Because the syringe flange 327 is trapped in the recess 317, the syringe body 324 cannot move relative to the tissue transfer device 310. When a vacuum is created at the inlet 316, the syringe plunger 322 is drawn up into the interior of the body 311 of the device 310. This action creates a vacuum in turn in the interior 325 of the syringe body 324. If the syringe inlet 326 is in contact with a tissue or fluid source, the tissue or fluid will be drawn up into the syringe body 324.

With the system 300 configured as shown in FIG. 4, tissue can be expelled from the syringe body 324 by applying pressurized gas at the inlet 316. The pressurized gas will cause the syringe plunger 322 to bear down on the tissue or fluid in the syringe body 324 and expel the tissue or fluid through the inlet 326. In some embodiments, the pressurized gas can be provided through a highly portable means such as a CO₂ canister or a small pump. In such an embodiment, the tissue handling system 300 need not be tethered to a bench-top but could be used in situations requiring maximum mobility such as an operating room.

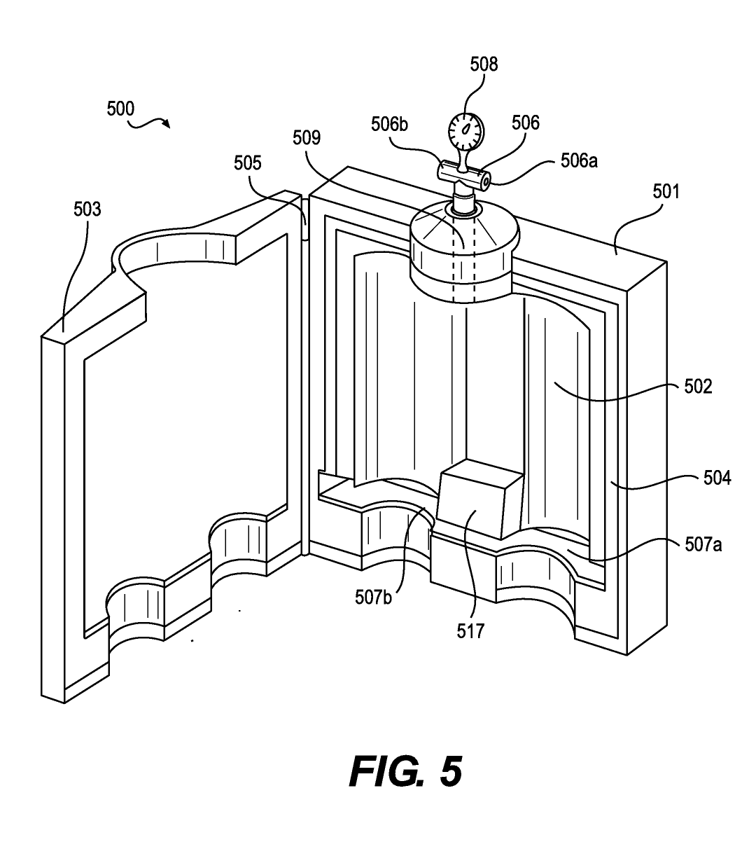

FIG. 5 depicts a tissue transfer device 500 that can simultaneously accommodate multiple tissue receptacles according to various embodiments of the present invention. The tissue transfer device 500 can include a body 501 that includes a chamber 502. An inlet 506 is in fluid communication with the interior volume of the chamber 502. When the tissue transfer device 500 is coupled to one or more tissue receptacles such as syringes, a change in the air pressure inside the chamber 502 can move an element of the tissue receptacles such as plungers or pistons.

The body 501 of the tissue transfer device 500 can be made of any suitable material that meets application-specific requirements. Such materials can include, but are not limited to, plastics, metals, and ceramics. In some embodiments, the body can include separate pieces, and one of the pieces may be a lid 503. The attachment mechanism between the lid 503 and other pieces of the body 501 can include hinges 505 of various designs. Alternatively, the lid 503 can attach to other pieces of the body 501 using a sliding friction fit or other suitable attachment methods.

In accordance with various embodiments, the body 501 can include a sealing gasket 504 that creates a seal around at least a portion of the chamber 502. In some embodiments, the sealing gasket 504 can be an o-ring or other structure made of various non-porous materials or one or more non-porous surfaces that press together to form a seal. To facilitate the seal, the body 501 may include a clasp or locking mechanism to hold the lid 503 tight to other pieces of the body 501. In some embodiments, reduced air pressure inside the chamber 502 can facilitate initial sealing at the sealing gasket 504 by holding fast the pieces of the body 501 including the lid 503. The sealing gasket 504 can form a seal among the pieces of the body 501 or between and among the body 501, lid 503, and elements of a tissue receptacle such as a syringe including a syringe body. The interior of the body 501 can include two or more recesses 507 that are shaped to accept at least a portion of one or more tissue receptacles. In some embodiments, the recesses 507 are shaped to accommodate a flange of a syringe. The body 501 may also include a protrusion 517 that extends from the wall of the body into the interior of the body. The recess 507 and protrusion 517 can work in concert to stabilize the main portions of two or more tissue receptacles and hold them motionless as the pistons or plungers move due to changes in air pressure inside the chamber 502.

The inlet 506 can be disposed at any location on the body 501 that does not interfere with passage of a piston or plunger through the body 501. In a preferred embodiment, the inlet 506 is located on the body 501 opposite to the entry points of plungers or pistons in a similar arrangement to intake and exhaust valves in a standard engine cylinder. The inlet 506 can be connected directly to the chamber 502 or can be in fluid communication with the chamber via a lumen 509. In some embodiments, two or more inlets 506 can exist on the body 501. In some embodiments, the chamber 502 can be subdivided into multiple chambers where each chamber is individually associated with an individual inlet 506. In such an embodiment, the loading or unloading of tissue from each tissue receptacle can be performed independently.

Each inlet 506 can include one or more ports 506a, 506b. In accordance with various embodiments, the port(s) 506a, 506b may be shaped or terminated to facilitate connection of pressure or vacuum sources. For example, the port(s) 506a, 506b could be a plastic through-port, a luer-type connector, a threaded connector, a swage fitting, or a pressure-fit connector. The pressure source attached to a port 506a, 506b can include a pressurized gas canister, a house source of medical compressed gas provided by a facility, or a mechanical pump. The vacuum source attached to a port 506a, 506b can include a mechanical pump or house vacuum provided by a facility. In preferred embodiments, a pressure regulator 508 is placed in the line between the pressure or vacuum source and the inlet 506. The pressure regulator 508 can provide a steady and reliable level of high or low pressure to allow smooth motion of the pistons or plungers within the body 501. The use of a set pressure or vacuum level prevents excessive shear forces on the tissue, which is known to reduce cell viability. In some embodiments, the pressure regulator 508 can be designed to include preset pressures for different sized tissue receptacles or cannulas or for different procedures. For example, the pressure regulator 508 may be set to 31 psi or less when the tissue receptacles are 60 cc syringes. The use of an air pressure/vacuum source and pressure regulator 508 can create a constant level of pressure/vacuum in the chamber 502 that, in turn, provides continuous motion of the pistons or plungers throughout a transfer operation. In other words, avoiding fluctuations in pressure in the chamber 502 can prevent unwanted changes in velocity of the piston or plunger particularly at the beginning or end of a tissue transfer operation.

In an embodiment, the pressure regulator 508 of the device 500 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied within the chamber 502 of the device 500. The memory of the computing device may include lookup tables or processor-executable instructions to ascertain a safe operating pressure range based on the user input(s). In some embodiments, the computing device may prevent a user from exceeding a preset maximum flow velocity and/or shear rate.

In some embodiments, the tissue transfer device 500 can act on two or more tissue receptacles that are not identical. For example, the tissue receptacles can be different shapes or sizes or can enclose different volumes. In some embodiments, the tissue receptacles can have different amounts of tissue within them at the start of an unloading operation. In such an embodiment, the constant pressure provided by using a pressure regulator can cause the tissue receptacles to each expel tissue at a constant rate. If one tissue receptacle empties and the piston or plunger can no longer move, the remaining pistons or plungers for the remaining tissue receptacles can still expel tissue at a constant rate without interruption.

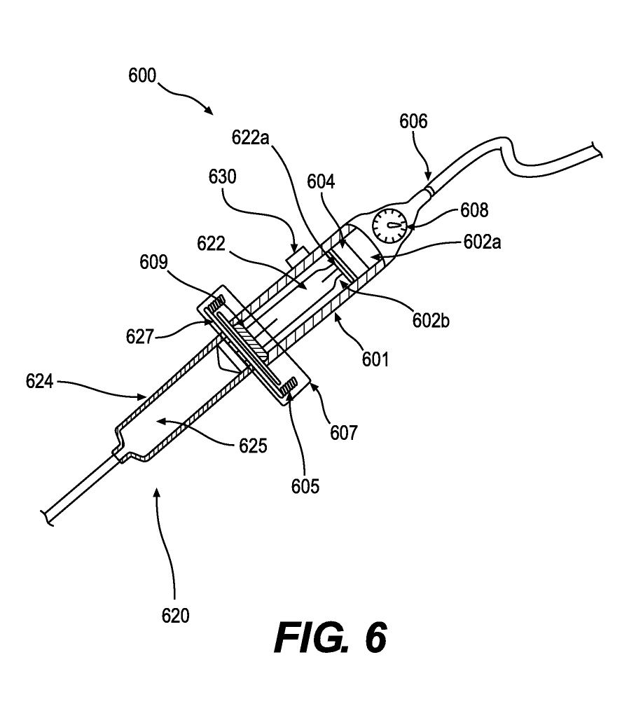

A tissue transfer device 600 is depicted in FIG. 6 attached to a syringe 620. The device 600 can include an inlet 606 and a body 601 having first and second chambers 602a, 602b. The device 600 can include an adaptor 607 and gasket 609 to attach the syringe 620 and seal the chamber 602. The device 600 can also include a button 630 to operate the high or low pressure sources directly from the device 600. When sealed, changing the air pressure within the first chamber 602a using either a high or low pressure source can cause a plunger 604 to move within the body 601 of the device 600. When low pressure is applied to the first chamber 602a, the plunger 604 moves within the body to create a vacuum in the second chamber 602b between the plunger 604 and the gasket 609 thus advancing a syringe plunger 622 into the interior of the body 601 and drawing tissue into the interior 625 of the syringe body 624. When high pressure is applied to the first chamber 602a, the plunger 604 applies pressure to a head 622a of the syringe plunger 622 thus advancing the syringe plunger 622 out from the interior of the body 201 and expelling tissue from the interior 625 of the syringe body 624.

The body 601 of the tissue transfer device 600 can be made of any suitable material that meets application-specific requirements. Such materials can include, but are not limited to, plastics, metals, and ceramics. In some embodiments, the body 601 of the tissue transfer device 600 may be transparent. In accordance with various embodiments, the diameter of the body 601 of the tissue transfer device 600 is approximately equal to a maximum diameter of a head 622a of the syringe plunger 622. In some embodiments, the diameter of the body 601 of the device 600 is larger than a maximum diameter of the head 622a of syringe plunger 622 receptacle to thereby increase the syringe filling or injection force. Increased filling or injection forces can improve efficiency of operation with respect to extremely viscous fluids. The button 630 of the device can be a three-way switch, a momentary-on switch, or two separate buttons to independently operate the negative and positive pressure sources themselves or valves connected to the sources.

The device 600 can include a coupler 607 to engage the syringe 620. The coupler 607 can include a gasket 609 and an attachment mechanism such as threads 605. In some embodiments, a seal is formed at the gasket 609 and the surface of a flange 627 of the syringe 620. A tight seal is secured by screwing the flange 627 of the syringe 620 into the threads 605 of the coupler 607. Alternatively, other attachment mechanisms can be used including, but not limited to, quick-release coupling, clamping, adhesion, or any other suitable method or device.

In accordance with various embodiments, the gasket 609 and the plunger 604 can create seals at the ends of the second chamber 602b and between the first and second chambers 602a, 602b. In some embodiments, the gasket 609 can include an o-ring or other structure made of various non-porous materials or may include a non-porous surface that presses against a flange 627 of the syringe 620 to form a seal. The plunger 604 can be made of rubber, polymers, or other suitable materials that will form a seal against the inner surface of the body 601. In accordance with various embodiments, the plunger 604 is long enough that it is unable to rotate within the interior of the body 601. As described previously with reference to the embodiments of FIG. 2, the plunger 604 may include an attachment mechanism that can engage with the head 622a of the syringe plunger 622.

The inlet 606 can include one or more ports. In accordance with various embodiments, the port or ports may be shaped or terminated to facilitate connection of pressure or vacuum sources. For example, the port(s) could be a plastic through-port, a luer-type connector, a threaded connector, a swage fitting, or a pressure-fit connector. As described above with reference to the embodiment of FIG. 1, the pressure source attached to a port can include a pressurized gas canister, a house source of medical compressed gas provided by a facility, or a mechanical pump. The vacuum source attached to a port can include a mechanical pump or house vacuum provided by a facility. In preferred embodiments, a pressure regulator 608 is placed in the line between the pressure or vacuum source and the inlet 606. The pressure regulator 608 can provide a steady and reliable level of high or low pressure to allow smooth motion of the syringe plunger 622 within the body 601. The use of a set pressure or vacuum level prevents excessive shear forces on the tissue, which is known to reduce cell viability. In some embodiments, the pressure regulator 608 can be designed to include preset pressures for different sized tissue receptacles or cannulas or for different procedures. For example, the pressure regulator may be set to 31 psi or less when the tissue receptacle is a 60 cc syringe. The use of an air pressure/vacuum source and pressure regulator 608 can create a constant level of pressure/vacuum in the first chamber 602a that, through its effect on the second chamber 602b, provides continuous motion of the syringe plunger 622 throughout a transfer operation. In other words, avoiding fluctuations in pressure in the first chamber 602a can prevent unwanted changes in velocity of the syringe plunger 622 particularly at the beginning or end of a tissue transfer operation.

In an embodiment, the pressure regulator 608 of the device 600 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied within the chamber 602a of the device 600. The memory of the computing device may include lookup tables or processor-executable instructions to ascertain a safe operating pressure range based on the user input(s). In some embodiments, the computing device may prevent a user from exceeding a preset maximum flow velocity and/or shear rate.

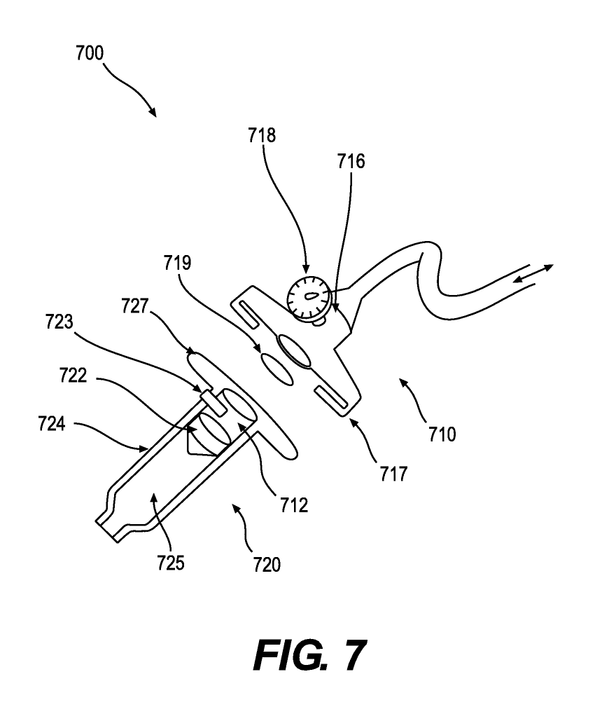

A different embodiment of a tissue transfer device 700 is depicted in FIG. 7. The device 700 can include a reusable portion 710 and a disposable portion 720. The reusable portion 710 can include an inlet 716 and a coupler 717 that can releasably engage with the disposable portion 720. The disposable portion 720 can include a body 724 enclosing an interior volume 725. A plunger 722 can move longitudinally within the body 724. A flange 727 can engage with the coupler 717, and a gasket 719 can provide a seal between the reusable portion 710 and the disposable portion 720. When the disposable portion 720 is attached to the reusable portion 710, a high or low pressure provided at the inlet 716 will cause the plunger 722 to advance into or out of the interior volume 725 of the disposable portion 720. The motion of the plunger 722 can draw tissue into or expel tissue out of the interior volume 725.

The body 724 of the disposable portion 720 can be made of any suitable material that meets application-specific requirements. Such materials can include, but are not limited to, plastics, metals, and ceramics. In some embodiments, the body 724 of the disposable portion 720 may be transparent. Once used, the disposable portion 720 can be discarded and a new, sterile disposable portion 720 can be attached to the reusable portion 710 to perform a new tissue transfer operation. Because the reusable portion 710 does not come into contact with tissue or fluids, it may be re-attached to a new disposable portion 720 with minimal need for cleaning or sterilization.

The device 700 can include a coupler 717 on the reusable portion 710 to engage the flange 727 of the disposable portion 720. The mechanism of the coupler 717 can be of any type that meets application-specific requirements including, but not limited to, quick-release, screw threads, clamps, temporary adhesives, manual pressure applied by a user, or any other suitable mechanism. A gasket 719 can be used to create a seal between the reusable portion 710 and the disposable portion 720.

In accordance with various embodiments, a portion of the interior volume 725 of the disposable portion 720 and the interior of the reusable portion 710 form a chamber 712 with the help of a gasket 719. In some embodiments, the gasket 719 can include an o-ring made of various non-porous materials or may include a non-porous surface or surfaces that presses between the flange 727 and the coupler 717. The plunger 722 can be made of rubber, polymers, or other suitable materials that will form a seal against the inner surface of the body 724 of the disposable portion 720. In accordance with various embodiments, the plunger 722 is long enough that it is unable to rotate within the interior volume 725 of the body 724. In some embodiments, the disposable portion 720 can include a stop 723 that retains the plunger 722 within the interior volume 725 of the disposable portion 720.

The inlet 716 can include one or more ports. In accordance with various embodiments, the port or ports may be shaped or terminated to facilitate connection of pressure or vacuum sources. For example, the port(s) could be a plastic through-port, a luer-type connector, a threaded connector, a swage fitting, or a pressure-fit connector. As described above with reference to the embodiment of FIG. 1, the pressure source attached to a port can include a pressurized gas canister, a house source of medical compressed gas provided by a facility, or a mechanical pump. The vacuum source attached to a port can include a mechanical pump or house vacuum provided by a facility. In preferred embodiments, a pressure regulator 718 is placed in the line between the pressure or vacuum source and the inlet 716. The pressure regulator 718 can provide a steady and reliable level of high or low pressure to allow smooth motion of the plunger 722 within the body 724. The use of a set pressure or vacuum level prevents excessive shear forces on the tissue, which is known to reduce cell viability. In some embodiments, the pressure regulator 718 can be designed to include preset pressures for different sized tissue receptacles or cannulas or for different procedures. The use of an air pressure/vacuum source and pressure regulator 718 can create a constant level of pressure/vacuum in the chamber 712 that provides continuous motion of the plunger 722 throughout a transfer operation. In other words, avoiding fluctuations in pressure in the chamber 712 can prevent unwanted changes in velocity of the plunger 722 particularly at the beginning or end of a tissue transfer operation.

In an embodiment, the pressure regulator 718 of the device 710 can be controlled by a computing device having a processor and a memory. The computing device can accept input from a user including, but not limited to, desired tissue transfer speed, maximum allowable shear force, aliquot amount, or physical data such as cannula diameter, cannula length, syringe body diameter, syringe volume, and tissue viscosity. The computing device may be operatively coupled to the pressure regulator or outlet to control the positive or negative pressure applied at the inlet 716 of the device 700. The memory of the computing device may include lookup tables or processor-executable instructions to ascertain a safe operating pressure range based on the user input(s). In some embodiments, the computing device may prevent a user from exceeding a preset maximum flow velocity and/or shear rate.

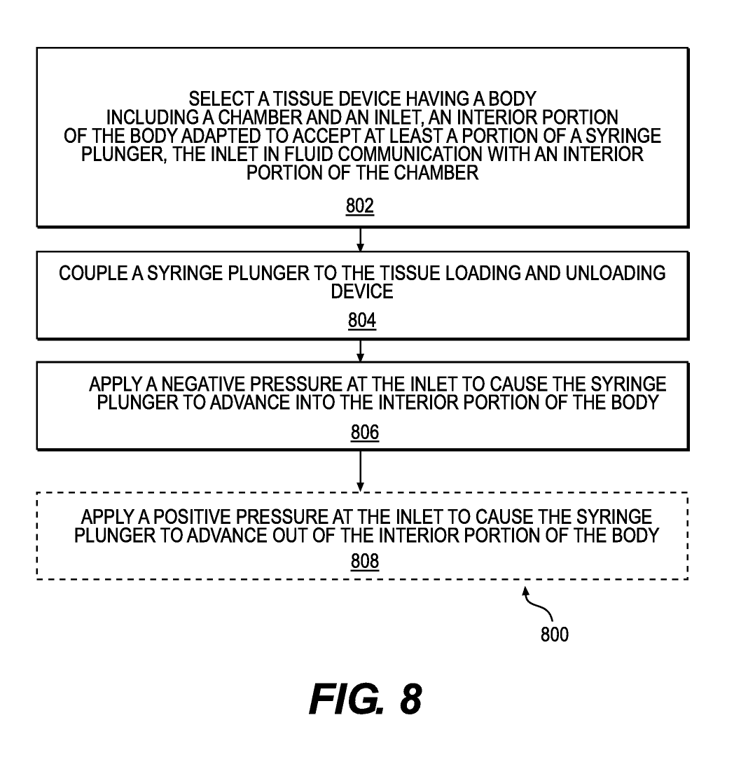

FIG. 8 presents a method 800 of transferring tissue according to various embodiments of the present invention. The method 800 includes a step 802 of selecting a tissue transfer device having a body including a chamber and an inlet. An interior portion of the body is adapted to accept at least a portion of a syringe plunger. The inlet is in fluid communication with an interior portion of the chamber. The method 800 also includes a step 804 of coupling a syringe plunger to the tissue transfer device. The method 800 also includes a step 806 of applying a negative pressure at the inlet to cause the syringe plunger to advance into the interior portion of the body. The method 800 also includes an optional step 808 of applying a positive pressure at the inlet to cause the syringe plunger to advance out of the interior portion of the body.

The method 800 will now be described in greater detail with reference to the embodiments depicted in previous figures. The step 802 of selecting a tissue transfer device having a body including a chamber and an inlet can include, but is not limited to, selecting a tissue transfer device 310 having a body 311 including a chamber 312 and an inlet 316 as described above with reference to FIG. 3. The step 804 of coupling a syringe plunger to the tissue transfer device can include, but is not limited to, placing a syringe 320 within the tissue transfer device 310 and securing the lid 313 so that the syringe plunger 322 is within the body 311 of the tissue transfer device 310 as described above with reference to FIG. 3.

The step 806 of applying a negative pressure at the inlet to cause the syringe plunger to advance into the interior portion of the body can include, but is not limited to, applying a negative pressure at a port 316a of the inlet 316 to cause the syringe plunger 322 to advance into the interior portion of the body 311 as described above with reference to FIGS. 3 and 4. The optional step 808 of applying a positive pressure at the inlet to cause the syringe plunger to advance out of the interior portion of the body can include, but is not limited to, applying a positive pressure at a port 316b of the inlet 316 to cause the syringe plunger 322 to advance out of the interior portion of the body 311 as described above with reference to FIGS. 3 and 4.

While the present invention has been described herein in conjunction with preferred embodiments, a person of ordinary skill in the art can effect changes, substitutions or equivalents to the systems and methods described herein that are intended to fall within the appended claims and any equivalents thereof.

1. A tissue transfer device, comprising:
a body including a chamber, an interior portion of the body adapted to accept at least a portion of a plunger of a syringe; and
an inlet in fluid communication with an interior portion of the chamber,
wherein a positive or negative pressure applied at the inlet causes the plunger of the syringe to advance into or out of the interior portion of the body.

2. The device of claim 1, wherein the inlet comprises a first port.

3. The device of claim 2, wherein the first port is connected to a source of negative pressure.

4. The device of claim 2, wherein the inlet further comprises a second port.

5. The device of claim 4, wherein the second port is connected to a source of positive pressure.

6. The device of claim 1, further comprising a sealing gasket to seal the chamber.

7. The device of claim 1, wherein a portion of the plunger of the syringe lies within the interior portion of the chamber.

8. The device of claim 1, further comprising a stopper within the body that can removably engage a head of the plunger of the syringe.

9. The device of claim 8, wherein a flange of the syringe abuts a distal end of the body of the device when the negative pressure is applied at the inlet and the plunger of the syringe is engaged with the stopper.

10. The device of claim 1, further comprising an actuation mechanism for control of application of the positive or negative pressure.

11. The device of claim 10, wherein the actuation mechanism comprises one or more buttons on the body.

12. The device of claim 10, wherein the actuation mechanism is foot-operated.

13. The device of claim 1, wherein the interior portion of the body is further adapted to accept at least a portion of a plunger of a second syringe.

14. The device of claim 13, wherein the positive or negative pressure applied at the inlet causes both plungers to advance into or out of the interior portion of the body.

15. The device of claim 1, further comprising a pressure regulator to limit the positive or negative pressure to a range that will not cause damage to a tissue.

16. The device of claim 15, further comprising a computing device including a processor and a memory, the computing device operatively coupled to the pressure regulator to control the positive or negative pressure and thereby control the speed of transfer of and force exerted on a tissue.

17. A tissue transfer device, comprising:
a body including a chamber including an outer wall and an interior portion contained within the outer wall;
a plunger contained at least partially within the interior portion; and
an inlet in fluid communication with an interior portion of the chamber,
wherein the device is configured such that a positive or negative pressure applied at the inlet causes the plunger to move within the interior portion of the body.

18. The device of claim 17, wherein the inlet comprises a first port.

19. The device of claim 18, wherein the first port is connected to a source of negative pressure.

20. The device of claim 18, wherein the inlet further comprises a second port.

21. The device of claim 20, wherein the second port is connected to a source of positive pressure.

22-31. (canceled)

32. A method of transferring tissue comprising:
selecting a tissue transfer device having a body including a chamber and an inlet, an interior portion of the body adapted to accept at least a portion of a plunger of a syringe, the inlet in fluid communication with an interior portion of the chamber;
coupling the plunger of the syringe to the tissue transfer device;
applying a negative pressure at the inlet to cause the plunger of the syringe to advance into the interior portion of the body.

33. The method of claim 32, further comprising applying a positive pressure at the inlet to cause the plunger of the syringe to advance out of the interior portion of the body.
